(19) United States
(12) Patent Application Publication
(10) Pub. No.: US 2009/0204014 A1
(43) Pub. Date: Aug. 13, 2009

(54) DOWN-SIZED SINGLE DIRECTIONAL RESPIRATORY AIR FLOW MEASURING TUBE

(76) Inventors: Eun Jong Cha, Chungcheongbuk-Do (KR); Kyung Ah Kim, Chungcheongbuk-Do (KR)

Correspondence Address: GREER, BURNS & CRAIN, 300 S WACKER DR, 25TH FLOOR, CHICAGO, IL, 60606, US

(21) Appl. No.: 12/095,434

(22) PCT Filed: Oct. 30, 2006
(86) PCT No.: PCT/KR06/04445

§ 371 Date: May 29, 2008

(51) Int. Cl. A61B 5/087

(52) U.S. Cl. 600/538

(57) ABSTRACT

A down-sized single directional respiratory air flow measuring tube is provided that is suitable for an electronic respiratory air flow measuring device such that the patients perform a self-test. The measuring tube includes: a cylindrical pipe including an inlet making contact with a mouth of an examinee and an outlet facing the inlet, in which the cylindrical pipe includes disposable material; and a detection rod positioned near the outlet, expanding out of a lower portion of the cylindrical pipe while passing through the cylindrical pipe from an upper portion of the cylindrical pipe, and formed with a closed upper portion, in which the detection rod has a plurality of sampling holes to measure dynamic pressure of a respiratory air flow at the first side of the inlet of the cylindrical pipe such that air flow between the center portion and a wall surface of the cylindrical pipe is measured.

### TECHNICAL FIELD

The present invention relates generally to a respiratory tube for a portable respiratory air flow measuring device. More particularly, the present invention relates to a down-sized respiratory tube for a portable respiratory air flow measuring device suitable for an electronic respiratory air flow measuring device enabling patients having chronic respiratory disease such as asthma to perform self-diagnostics.

### BACKGROUND ART

When all kinds of respiratory function tests such as spirometry are performed, a respiratory air flow is essentially measured. The measurement of the respiratory air flow is a kind of clinical examination in which the change signals of a lug volume according to the breathing of a patient are continuously recorded, and then the recorded signals are analyzed.

Recently, most widely used respiratory air flow measuring schemes include pneumotachography and tubinometry. In the above respiratory air flow measuring schemes, a sensor device must be positioned on a respiratory path to convert a respiratory air flow into measurable physical parameters. For example, according to the respiratory air flow measuring scheme of a pneumotachography, a fluid resistance member is positioned on the center portion of a respiratory tube forming a respiratory path, and the difference in static pressure obtained at both sides of the fluid resistance member is measured to measure the respiratory air flow.

In a respiratory air flow measuring device employing the pneumotachography, since a fluid resistance member is positioned on a respiratory path of an examinee (who are subject to examination) to interrupt the breathing of the examinee, a flow rate signal representing a respiratory function of the examinee is changed so that examination reliability may be degraded. In addition, since the structure of the fluid resistance member positioned on the respiratory path is very complex and requires a great amount of the manufacturing costs, a disposable respiratory tube having the fluid resistance member attached thereto is hard to be manufactured. Accordingly, when a plurality of examinees is subject to respiratory function tests, the examinees may be infected with their disease.

In order to overcome the problem of the respiratory air flow measuring device employing the pneumotachography, a new respiratory air flow measuring device is developed to measure the respiratory air flow by measuring a dynamic pressure instead of a static pressure.

FIG. 1 is a view showing a principle of measuring a respiratory air flow by using the dynamic pressure.

As shown in FIG. 1, a respiratory tube 1 for measuring a respiratory air flow by using dynamic pressure is provided at the center portion with pitot tubes 2 and 2 symmetrical to each other such that differential pressure is measured when a user exhales or inhales. The respiratory tube 1 for measuring a respiratory air flow by using dynamic pressure is based on Bernoulli's principle in which the sum of the kinetic energy and the potential energy of the respiratory air flow is constant when the respiratory air flow passes through the respiratory tube 1. In other words, when the respiratory air flow is measured by using dynamic pressure, the velocity (u) of bidirectional air flow is expressed as shown in Equation 1.

$$\begin{matrix}\begin{matrix}u ≡ uL - uR ∝ \pm \sqrt{{P}_{L} - {P}_{R}} \\ = S \cdot \sqrt{{P}_{D}}\end{matrix} & < Equation 1 >\end{matrix}$$

In Equation 1,

### S, P_{D}, u, uL, and uR

denote a proportional constant, dynamic pressure

### (P_{L}−P_{R}),

the velocity of an air flow, the velocity of an expiratory flow, and the velocity of an inspiratory flow.

In Equation 1, when a respiratory air flow is measured by using dynamic pressure, since the pitot tubes 2 and 2′ are symmetrical to each other, potential energy components are canceled to each other, and the differential pressure

### (P_{L}−P_{R})

derived from the expiratory flow and the inspiratory flow reflects the dynamic pressure

### (P_{D}).

Equation 1 represents that the respiratory air flow is proportional to the square root

### (√P_{D})

of the dynamic pressure, and one proportional constant exists. Accordingly, the respiratory air flow measuring system using the dynamic pressure can solve the problems such as respiratory interruption due to a fluid resistance member and a complex structure of the fluid resistance member in compared to the above respiratory air flow measuring device employing a pneumotachography.

Korean Patent Registration No. 10-0432640-0000 issued to applicant of the present invention discloses the above respiratory air flow measuring device using dynamic pressure. As shown in FIGS. 2 and 3, the patent discloses a detection rod 240 inserted into a cylindrical pipe 220 including paper such that the detection rod 240 is detachable from the cylindrical pipe 220. The cylindrical pipe 220 includes an inlet 222 and an outlet 224 facing the inlet 222, and a screen cap 260 in the form of a mesh is installed in the inlet 222 to stabilize the streamline of air. The detection rod 240, which is installed in the cylindrical pipe 220 to sample a respiratory air flow and then transform the respiratory air flow into dynamic pressure, includes two air tubes 242 communicating with a differential pressure sensor provided in a measurement module in vertical to a plurality of sampling holes 244.

In the above respiratory air flow measuring device issued to applicant of the present invention, the detection rod 240 has sampling holes with the same size so that the accuracy for the measurement of a respiratory air flow is enhanced and the manufacturing cost is reduced. In addition, a disposable respiratory air flow tube including paper is provided In the above respiratory air flow measuring device, so that problems related to infection between examinees are completely solved when the respiratory air flow of a plurality of examinees is measured.

Meanwhile, patients having chronic respiratory disease must do self-management in a trend in which the patients rapidly increase according to environmental pollution and industrialization. In the case of asthma that is a representative example of the chronic respiratory disease, a respiratory track of the patient is narrowed, respiratory distress is caused, and then the patient may die of asthma attack.

The patients having chronic respiratory disease typically carry out self-management by measuring a peak expiratory flow rate (PEF) twice every day. In this case, since a commonly used peak expiratory flow meter employed for the measurement of the PEF operates by the elasticity of a spring to measure only the PEF, the peak expiratory flow meter has a limitation in the self-management of the patients having the chronic respiratory disease. Parameters for forced vital capacity examination, such as forced vital capacity (FVC) and forced expiratory volume in 1 second (FEV 1.0) are very important for the actual self-management of the patients having the chronic respiratory disease. In addition, since, a expiratory flow waveform must be accumulated when the peak expiratory flow rate is checked, an electronic spirometer is required.

However, since a conventional respiratory air flow measuring device such as a clinical spirometer is manufactured for clinical examination, the respiratory air flow measuring device has a large size and a high price. Accordingly, it is actually impossible for the patients having chronic respiratory disease to measure their respiratory air flow while carrying with the respiratory air flow measuring device. In addition, the most difficulty when a portable electronic spirometer is down-sized exists in the down-sizing of a sensor for measuring a respiratory air flow and transforming vital parameters that cannot be directly measured into measurable physical parameters.

In addition, the conventional respiratory air flow measuring device employing the pneumotachography cannot be down-sized because a fluid resistance member must be inserted into a respiratory path (a respiratory tube), and the fluid resistance member includes a mesh screen, a capillary tube and the like. In addition, a respiratory air flow measuring device employing a tubinometry cannot be down-sized because a rotatable turbine is installed in the respiratory path (the respiratory tube).

### DISCLOSURE OF INVENTION

### Technical Problem

Accordingly, the present invention has been made keeping in mind the above problems occurring in the prior art, and an object of the present invention is to provide a down-sized single directional respiratory air flow measuring tube allowing patients having chronic respiratory disease to simply measure their respiratory air flow using the down-sized single directional respiratory air flow measuring tube by significantly reducing the diameter and the length of the down-sized single directional respiratory air flow measuring tube while preventing the breath of the patients from being interrupted in the down-sized single directional respiratory air flow measuring tube.

### Technical Solution

To accomplish these objects, according to one aspect of the present invention, there is provided a down-sized single directional respiratory air flow measuring tube comprising: a cylindrical pipe including an inlet making contact with a mouth of an examinee and an outlet facing the inlet, in which the cylindrical pipe includes disposable paper or disposable plastic; and a detection rod positioned closely to the outlet, expanding out of a lower portion of the cylindrical pipe while passing through the cylindrical pipe from an upper portion of the cylindrical pipe, formed in a shape of a tube having a closed upper portion and an opened lower portion, and formed with a plurality of sampling holes, which are used to measure air flow at a side of the inlet of the cylindrical pipe on a respiratory path of the cylindrical pipe and provided in a longitudinal direction.

### ADVANTAGEOUS EFFECTS

As described above, a down-sized single directional respiratory air flow measuring tube for a portable respiratory air flow measuring device according to the present invention is the most suitable for a portable respiratory air flow measuring device, which is used for self-management of a patient having chronic respiratory disease such as asthma, among portable medical appliances that are actively used recently. In addition, in the down-sized single directional respiratory air flow measuring tube according to the present invention, sensitivity is remarkably improved, the manufacturing cost is reduced, and a disposable material is used so that a plurality of patients is prevented from being infected with their diseases.

### BRIEF DESCRIPTION OF THE DRAWINGS

FIG. 1 is a view showing a principle of measuring a respiratory air flow by using dynamic pressure;

FIG. 2 is a view showing the structure of a respiratory tube disclosed in Korean Patent Registration No. 10-0432640-0000 issued to applicant of the present invention;

FIG. 3 is a view showing the structure of a detection rod inserted into the respiratory tube shown in FIG. 2;

FIG. 4 is a sectional view showing the structure of a down-sized single directional respiratory air flow measuring tube according to the present invention;

FIG. 5 is a block diagram showing the structure of a test device for measuring static pressure and dynamic pressure in order to determine the size of the down-sized single directional respiratory air flow measuring tube shown in FIG. 4;

FIG. 6 is a graph showing the correlation between the maximum respiratory air flow value and the diameter of the down-sized single directional respiratory air flow measuring tube obtained through the test device shown in FIG. 5; and

FIG. 7 is a graph showing the correlation between the dynamic pressure and the diameter of a down-sized single directional respiratory air flow measuring tube according to the present invention.

### BEST MODE FOR CARRYING OUT THE INVENTION

Hereinafter, exemplary embodiments of the present invention will be described in detail with reference to accompanying drawings.

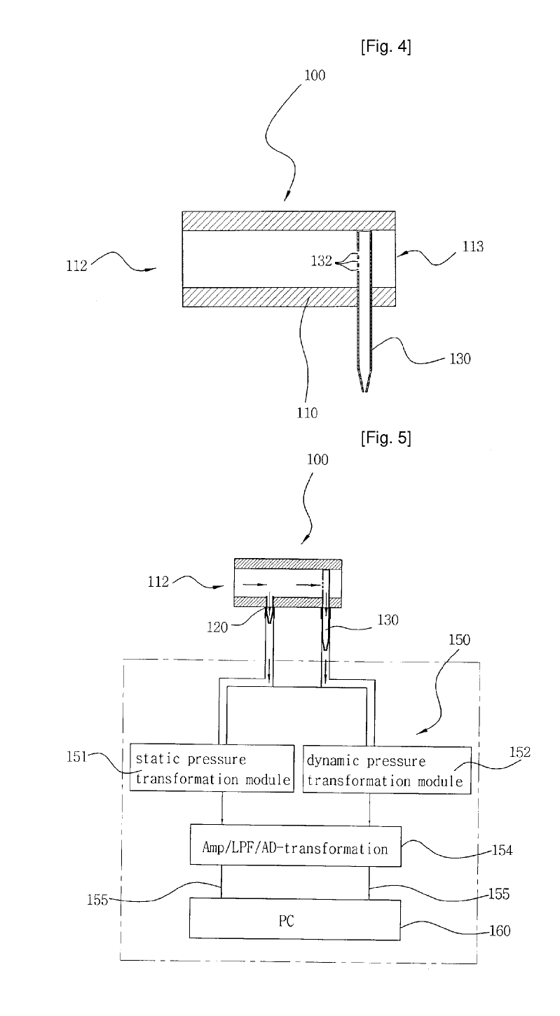

FIG. 4 is a sectional view showing the structure of a down-sized single directional respiratory air flow measuring tube 100 according to the present invention. FIG. 5 is a block diagram showing the structure of a test device for measuring static pressure and dynamic pressure in order to determine the standard of the down-sized single directional respiratory air flow measuring tube 100 shown in FIG. 4.

Referring to FIG. 4, the down-sized single directional respiratory air flow measuring tube 100 according to the present invention includes disposable paper or disposable plastic, and comprises a cylindrical pipe 110 and a detection rod 130. The cylindrical pipe 110 includes an inlet 112 making contact with the mouth of an examinee (who are subject to examination) and an outlet 113 facing the inlet 112. The detection rod 130 is provided in close to the outlet 113 of the cylindrical pipe 110 and includes a slim rod tube having the inner diameter of 1 mm.

The detection rod 130 is provided in close to the outlet 113 of the cylindrical pipe 110 within a distance of 5 mm from the outlet 113 of the cylinder-type tube 110. The detection rod 130 includes a rod-type circular tube which has the inner diameter of 1 mm and expands out of the lower portion of the cylindrical pipe 110 while passing through the cylindrical pipe 110 from the upper portion of the cylindrical pipe 110. The detection rod 130 has a closed upper portion and an opened lower portion. A plurality of sampling holes 132 are formed at the first side of the detection rod 130 in the cylindrical pipe 110, that is, at the side of the inlet of the cylindrical pipe 110 and spaced apart from each other by a predetermined distance in a longitudinal direction of the detection rod 130 in order to measure a flow rate.

In this case, the cylindrical pipe 110 of the down-sized single directional respiratory air flow measuring tube 100 has a length of 35 mm and a diameter of 15 mm, and only the detection rod 130, which is a rod-shape circular tube having the inner diameter of 1 mm, is positioned inside the cylindrical pipe 110 forming a respiratory path of the down-sized single directional respiratory air flow measuring tube 100, so that fluid resistance rarely exists. In addition, three sampling holes 132 provided at the first side of the detection rod 130 (the side of the inlet 112 of the cylindrical pipe 100) are positioned on the central axis through which the air flows and in the regions spaced apart from the central axis by the distances of ±2.5 mm, respectively.

The length of the cylindrical pipe 110 constituting the down-sized single directional respiratory air flow measuring tube 100 is set as the minimum length of 35 mm allowing an examinee to easily hold the cylindrical pipe 110 in his/her mouth and breath, and allowing the detection rod 30 for measuring a flow rate to be inserted into the cylindrical tub 110. If the length of the cylindrical pipe 110 is set, the diameter of the cylindrical pipe 110 and the installation position of the detection rod 130 are determined according to the set length, and the diameter of the cylindrical pipe 110 must satisfy the standard of American Thoracic Society (ATS).

The ATS recommends that the maximum value of fluid resistance in a clinical spirometer is 1.5 cmH2O/l, and the maximum value of fluid resistance in a spirometer for self-diagnostics is 2.5 cmH2O/l. In addition, the ATS recommends that the maximum respiratory air flow value must be 14 l/sec.

The fluid resistance of the down-sized single directional respiratory air flow measuring tube 100 may be calculated by measuring the static pressure (Ps) of a fluid flowing through the down-sized single directional respiratory air flow measuring tube 100. The static pressure (Ps) is expressed by multiplying fluid resistance R by respiratory air flow (F) as shown in the following Equation 2.

P_{S}=R·F Equation 2

If the maximum values of the fluid resistance (R) and the respiratory air flow (F) recommended by the ATS are multiplied by each other through Equation 2, an allowable value of the static pressure (Ps) is calculated. Through Equation 2, the allowable value of the static pressure (Ps) of a respiratory pipe is obtained as 21 cmH2O in the clinical spirometer, and obtained as 35 cmH2O in the spirometer for self-diagnostics.

As shown in FIG. 5, in order to measure the static pressure (Ps) of the down-sized single directional respiratory air flow measuring tube 100 according to the present invention, a static pressure measuring pipe 120 is additionally provided, in which the static pressure measuring pipe 120 is a rod-shape circular tube having the inner diameter of 1 mm that is installed in vertical to the cylindrical pipe 110 while communicating with the lower portion of the cylindrical pipe 110 at a position spaced apart from the inlet 112 of the cylindrical pipe 110 by a distance of 5 mm to measure the static pressure Ps of a respiratory air flow passing through the down-sized single directional respiratory air flow measuring tube 100.

As shown in FIG. 5, a test device for determining the standard of the down-sized single directional respiratory air flow measuring tube 100 according to the present invention includes a measurement module 150, which is connected to the down-sized single directional respiratory air flow measuring tube 100 provided with the static pressure measuring pipe 120 at a position spaced apart from the inlet 112 by the distance of 5 mm, in order to perform a test.

The measurement module 150 includes a static pressure transformation module 151 and a dynamic pressure transformation module 152. The static pressure transformation module 151 is connected to the static pressure measuring pipe 120 additionally installed in the down-sized single directional respiratory air flow measuring tube 100 to transform static pressure representing potential energy of a respiratory air flow into an electrical signal. The dynamic pressure transformation module 152 is connected to the detection rod 130 of the down-sized single directional respiratory air flow measuring tube 100 according to the present invention to transform dynamic pressure of the respiratory air flow obtained from the detection rod 130 into an electrical signal. The static pressure transformation module 151 and the dynamic pressure transformation module 152 include a typical pressure sensor.

The electrical signals output from the static pressure transformation module 151 and the dynamic pressure transformation module 152 are amplified according to a typical amplification scheme, and then the noises of the electrical signals are filtered through a low pass filter (LPF). Thereafter, the electrical signals are converted into signals suitable for a test through an electrical circuit 154 for analog/digital (A/D) converting, transmitted to a computer 160 through interface connection lines 155 installed at the lower portion of the measurement module 150, and used for a test.

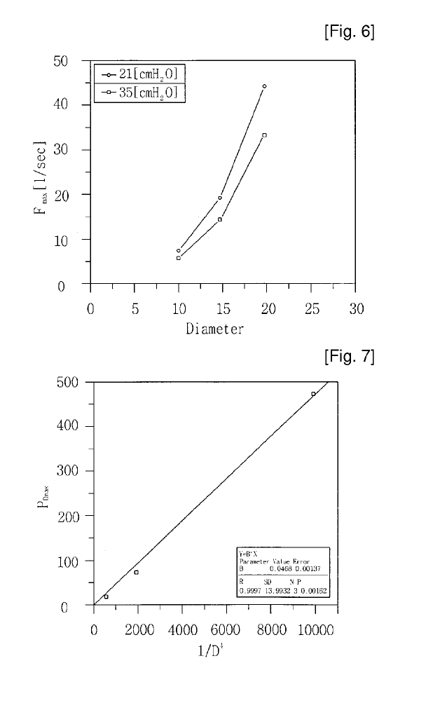

FIG. 6 is a graph showing the correlation between the maximum respiratory air flow value and the diameter of the down-sized single directional respiratory air flow measuring tube obtained through the test device, and FIG. 7 is a graph showing the correlation between the dynamic pressure and the diameter of the down-sized single directional respiratory air flow measuring tube according to the present invention.

The correlation between the static pressure Ps and the maximum respiratory air flow value (F) is measured according to the diameter (D) of the down-sized single directional respiratory air flow measuring tube 100 through the test device shown in FIG. 5, and the maximum respiratory air flow value (Fmax) measurable according to the diameter (D) of the down-sized single directional respiratory air flow measuring tube 100 is calculated by applying the maximum static pressure (Ps) value recommended in the ATS to Equation 2. FIG. 6 shows the calculation result for the maximum respiratory air flow value (Fmax), which is measurable according to diameter values (D) of the down-sized single directional respiratory air flow measuring tube 100, relative to the static pressure values (Ps) (21 cmH2O in a clinical spirometer, and 35 cmH2O in a spirometer for self-test) recommended by the ATS. FIG. 6 represents the maximum respiratory air flow value (Fmax) showing the maximum fluid resistance (R) allowed by the ATS according to diameters when the diameter of the down-sized single directional respiratory air flow measuring tube 100 is set.

Since the maximum measurement range of air flow standardized by the ATS corresponds to 0˜14l/sec (the maximum respiratory air flow value (Fmax) is less than or equal to 14l/sec), if the diameter (D) of the down-sized single directional respiratory air flow measuring tube 100 making the maximum respiratory air flow value of 14l/sec is calculated through an interpolation scheme, so that the diameter (D) is 14.7 mm in the clinical spirometer, and 12.8 mm in the self-diagnostics spirometer.

The diameter is the minimum diameter of the down-sized single directional respiratory air flow measuring tube 100 satisfying the standard of the ATS. Since the difference between the minimum diameters of the clinical spirometer and the self-diagnostics spirometer is only 1.91 mm, the minimum diameter of the down-sized single directional respiratory air flow measuring tube 100 is set as 15 mm according to the present invention.

As a result, the length of the down-sized single directional respiratory air flow measuring tube 100 according to the invention is set as 35 mm by taking account of convenience of examinee and the insertion of the detection rod. The minimum diameter is set as 15 mm by taking the standard of the ATS into consideration according to the length of 35 mm. In other words, when the length of the down-sized single directional respiratory air flow measuring tube 100 is 35 mm, the minimum diameter satisfying the standard of the ATS is 15 mm. If the length and the diameter are calculated as a volume, the volume is about 6.2 cm³. Accordingly, the down-sized single directional respiratory air flow measuring tube 100 has a size allowing it to be installed in a portable device.

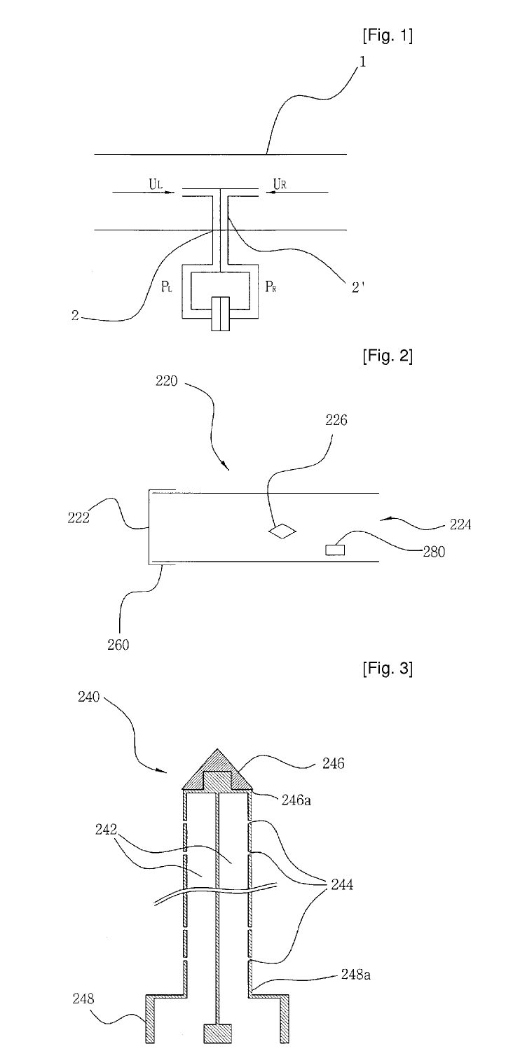

Further, in the down-sized single directional respiratory air flow measuring tube 100 according to the present invention, the respiratory air flow detected through the three sampling holes 132 of the detection rod 130 positioned on a respiratory path is transformed into the value of the dynamic pressure (P_{D}) by the pressure sensor such as the dynamic pressure measuring module 152 of FIG. 5 installed in the lower portion of the detection rod 130. The detection rod 130 of the down-sized single directional respiratory air flow measuring tube 100 according to the present invention is located at a position spaced apart from the outlet 113 of the cylindrical tube 110 by a distance of 5 mm. Since the pressure of the down-sized single directional respiratory air flow measuring tube 100 is relative based on atmospheric pressure, if the position of the detection rod 130 is close to external atmosphere, the potential energy component of a respiratory air flow is identical to the external atmosphere. Accordingly, since it is unnecessary to cancel the potential position component, the detection rod 130 of the down-sized single directional respiratory air flow measuring tube 100 detects only the dynamic pressure value (P_{D}). In other words, the respiratory tube can be fabricated in a simple structure at a low cost by using only one tube without measuring differential pressure using two pilot tubes to compensate for the potential position components as shown in FIG. 1.

If Equation 1 related to the value of the dynamic pressure (P_{D}) is modified, the dynamic pressure (P_{D}) is proportional to the second power of the velocity (u) of air flow as shown in Equation 3, the respiratory air flow F is obtained by multiplying the velocity (u) of the air flow by the area (A) of the down-sized single directional respiratory air flow measuring tube through a continuity principle as shown in Equation 4, and the sectional area of the down-sized single directional respiratory air flow measuring tube is expressed as shown in Equation 5. Accordingly, if the dynamic pressure (P_{D}) is calculated by simultaneously solving Equations 3, 4, and 5, Equation 6 is obtained.

$$\begin{matrix}{P}_{D} = S \cdot {u}^{2} & < Equation 3 > \\ F = A \cdot u & < Equation 4 > \\ A = \frac{π {D}^{2}}{4} & < Equation 5 > \\ {P}_{D} = 16 SF 2 / {π}^{2} \cdot 1 / D 4 ∝ 1 / D 4 & < Equation 6 >\end{matrix}$$

In the above Equations,

### A, S, P_{D}, u, and D

denote the sectional area of the down-sized single directional respiratory air flow measuring tube, a proportional constant, dynamic pressure, the velocity of air flow, and the diameter of the down-sized single directional respiratory air flow measuring tube.

Since the dynamic pressure (P_{D}) is proportional to 1/D4 in Equation 6, if the diameter (D) is changed by 10 cm to 1 cm for convenience, the measurement result of the dynamic pressure (P_{D}) actually represents a linear regression equation as shown in FIG. 7.

As shown in FIG. 6, when the diameter and the length of the down-sized single directional respiratory air flow measuring tube 100 according to the present invention is 15 mm and 35 mm, respectively, (1/D4=1975), the maximum dynamic pressure of about 75 cmH2O can be obtained. This signifies that sensitivity is improved by seven times or more of the maximum static pressure of 10 cmH2O obtained through a pnuemotach scheme widely used in clinic treatment.

In the down-sized single directional respiratory air flow measuring tube 100 according to the present invention, the structure of the detection rod 130 of transforming the velocity of a respiratory air flow into dynamic pressure is simplified so that only one-way expiratory flow is measured instead of bi-directional air flow. A forced vital capacity (FVC) examination, which is the most important and widely used of vital capacity examining items, is to analyze an expiratory flow signal obtained when an examinee exhales as much as possible to obtain various kinds of clinic diagnostic parameters. This is an examination to obtain mechanical characteristics of a respiratory appliance based on expiratory flow limitation in which the respiratory track of the examinee is narrowed as the examinee exhales, and most of diagnostic parameters are obtained from the expiratory flow signal.

In particular, since only five or less parameters that can be obtained from an expiratory flow signal are used for self-diagnostics of a patient having chronic respiratory disease, it is unnecessary to measure an inspiratory flow. Accordingly, the down-sized single directional respiratory air flow measuring tube 100 according to the present invention measures an expiratory flow detected through three sampling holes 132 provided in the front surface (in close to the inlet of the cylindrical pipe 110) of the down-sized single directional respiratory air flow measuring tube 100 while passing through a respiratory path. The expiratory flow is converted into an electrical signal representing the value of dynamic pressure in the dynamic pressure trans-formation module 152 that is a typical pressure sensor.

Since the detection rod 130 is a slim rod-type tube, fluid resistance rarely exists in the detection rod 130. In addition, since dynamic pressure increases as shown in Equation 6 if the diameter of the down-sized single directional respiratory air flow measuring tube 100 is narrowed, higher dynamic pressure can be obtained with respect to a predetermined air flow as the down-sized single directional respiratory air flow measuring tube 100 is scaled down. This signifies sensitivity improvement, in which measurement sensitivity is increased as the down-sized single directional respiratory air flow measuring tube 100 is down-sized, and a respiratory air flow measuring device can be manufactured by using a low-priced and small-sized pressure sensor.

Since fluid resistance of interrupting the breathing of an examinee is increased as the diameter of the down-sized single directional respiratory air flow measuring tube 100 is decreased, the fluid resistance becomes a restriction condition in downsizing of the down-sized single directional respiratory air flow measuring tube 100. However, since the detection rod 130 positioned on a respiratory path of the down-sized single directional respiratory air flow measuring tube 100 according to the present invention to measure dynamic pressure is only a slim rod-type circular tube having the inner diameter of 1 mm, the fluid resistance rarely exists in the detection rod 130.

Accordingly, the down-sized single directional respiratory air flow measuring tube 100 according to the present invention has the length of 35 mm set within the range that do not cause problems related to utility such that the down-sized single directional respiratory air flow measuring tube 100 is suitable for a portable respiratory air flow measuring device of a patient having chronic respiratory disease. Then, the measurement result of static pressure (Ps) which is obtained from the static pressure measuring pipe 120 of the down-sized single directional respiratory air flow measuring tube 100 and reflects fluid resistance, is analyzed according to diameters of the respiratory air flow measuring tube, so that the diameter of the down-sized single directional respiratory air flow measuring tube 100 is 15 mm or more.

As described above, a down-sized single directional respiratory air flow measuring tube for a portable respiratory air flow measuring device according to the present invention is the most suitable for a portable respiratory air flow measuring device, which is used for self-management of a patient having chronic respiratory disease such as asthma, among portable medical appliances that are actively used recently. In addition, in the down-sized single directional respiratory air flow measuring tube according to the present invention, sensitivity is remarkably improved, the manufacturing cost is reduced, and a disposable material is used so that a plurality of patients is prevented from being infected with their diseases.

Although a preferred embodiment of the present invention has been described for illustrative purposes, those skilled in the art will appreciate that various modifications, additions and substitutions are possible, without departing from the scope and spirit of the invention as disclosed in the accompanying claims.

1. A down-sized single directional respiratory air flow measuring tube comprising: a cylindrical pipe including an inlet making contact with a mouth of an examinee and an outlet facing the inlet, in which the cylindrical pipe includes disposable paper or disposable plastic; and a detection rod positioned closely to the outlet, expanding out of a lower portion of the cylindrical pipe while passing through the cylindrical pipe from an upper portion of the cylindrical pipe, formed in a shape of a tube having a closed upper portion and an opened lower portion, and formed with a plurality of sampling holes, which are used to measure air flow at a side of the inlet of the cylindrical pipe on a respiratory path of the cylindrical pipe and spaced apart from each other by a predetermined distance in a longitudinal direction.

2. The down-sized single directional respiratory air flow measuring tube as claimed in claim 1, wherein the cylindrical pipe has a length of 35 mm or less and a minimum diameter of 15 mm or more.

3. The down-sized single directional respiratory air flow measuring tube as claimed in claim 1, wherein the detection rod has an inner diameter of 1 mm or less.

4. The down-sized single directional respiratory air flow measuring tube as claimed in claim 1, wherein the detection rod is formed at a position spaced apart from the outlet of the cylindrical tube by a distance of 5 mm or less.
